(19) United States
(12) Patent Application Publication
(10) Pub. No.: US 2005/0182076 A1
(43) Pub. Date: Aug. 18, 2005

(54) Transdermal penetration system and treatment for cellulite

(75) Inventors: Adam A. Pacheco, Las Vegas, NV (US); Teresa Leigh Barr, Hood River, OR (US)

Correspondence Address: TERESA LEIGH BARR, 1908 ORCHARD ROAD, HOOD RIVER, OR, 97031, US

(73) Assignee: Adam A. Pachelo (ANO) TINA WEISS

(21) Appl. No.: 11/054,312

(22) Filed: Feb. 9, 2005

Related U.S. Application Data
(60) Provisional application No. 60/544,770, filed on Feb. 13, 2004.

(51) Int. Cl. A61K 31/522; A61K 31/455; A61K 31/4164

(52) U.S. Cl. 514/263.32; 514/355; 514/389

(57) ABSTRACT

The present invention is a powerful vasodilatation and transdermal penetration system, which quickly penetrates the dermis of the skin and deposits beneficial ingredients at the subcutaneous layer, which then stimulates the capillary blood flow to fat cells and increases the metabolizing process of fat cells and cellulite, thereby reducing the size of fat cells, thus creating a smoother appearance to the surface of the skin as well as reducing the appearance of the size of the affected area.

### CROSS REFERENCE TO RELATED APPLICATION

The present invention claims priority to U.S. Provisional Patent Application Ser. No. 60/544,770, filed in the United States Patent and Trademark Office on Feb. 13, 2004.

### BACKGROUND OF THE INVENTION

Cellulite is typically characterized by fatty deposits beneath the skin of the body of humans, namely women. Cellulite is a term given to the lumpy or grainy appearance to the surface of the skin in areas such as the thighs and buttocks, but not limited to the thighs and buttocks. Cellulite can also be found on the stomach, the back, arms, under the chin and breast area as well.

Researchers have studied the thighs and buttock areas of cadavers and performed biopsies, which have concluded that the subcutaneous tissue of the thighs is derived of three layers of fat or fat chambers with two connective tissue planes between them. The fat chambers on the average are 0.0-1.5 centimeters and are separated by a thin partition or membrane called septa, which can be described as dividing something into two or more cavities, such as the tissue separating the nostrils or internal dividing walls in the seed heads of poppies. The septa of connective tissue is called retinacula cutis. The retinacula cutis resembles a pattern that is arched and radial, and anchors to the overlaying corium, or dermis, (which is a thick layer of sensitive skin or connective tissue beneath the epidermis) that contains blood, lymph vessels, sweat glands, and nerve endings. Papillae adiposae, or small proturbances containing fat project from these chambers and into the corium, thus creating an orange peel, mattress-like or dimpled appearance.

A product that could transdermally penetrate the corium of the skin to stimulate the papillae adiposae within the retinacula cutis to effectively treat the symptoms of cellulite or fatty deposits under the skin would be beneficial.

The orange peel appearance or dimpling of the skin is a characteristically inherent to women. For men, the subcutaneous tissue is thinner, and has its own unique network, such as crisscrossing septa that divide the fat chamber into smaller polygonal units and the corium is thicker in thighs of men than women.

For these and other reasons, a need has existed for women to be able to decrease the appearance of cellulite on the body, as well as tone the skin without surgical procedures (such as liposuction or surgical removal) by using a cream, gel, lotion, enhanced oil, spray or the like that can easily and safely be used from 1 to 4 times per day.

Reduced capillary blood flow is a characteristic of cellulite. When capillary flow is decreased, so is the flow of lymph fluid. Consequently, tissue is depended upon to pump the fluid, as lymphatic capillaries have no way to pump the lymph fluid. The fatty mass of cellulite is aggravated by sluggish circulation, which slows down lipid metabolism and tends to increase the appearance of cellulite.

U.S. Pat. No. 6,394,946; Giovannini, et al., teaches topical application of an elastomeric device for the treatment of cellulite and is hereby incorporated by reference.

U.S. Pat. No. 5,705,170; Kong, et al., teaches an herbal cellulite treatment, wherein the invention provides a herbal cellulite treatment employing, in preferred embodiments, topical treatments, both method and cosmetic composition, wherein a refined lipophilic extract, and preferably also a refined aqueous extract of a Malvaceae plant, preferably whole Hibiscus Abelmoschus, are applied to the skin overlying cellulite-afflicted tissues. The treatments are intended to last at least four, and preferably eight or more weeks. Clinical tests show surprisingly superior results to those obtainable with aminophylline compositions. Inventive treatments can reduce thigh diameters and fatty layer thickness, as well as skin condition. In vitro tests show remarkable lipolytic properties, apparently attributable to beta-receptor stimulation, and valuable lipogenesis inhibition properties apparently attributable to .alpha.sub.2 blocking and is hereby incorporated by reference.

U.S. Pat. No. 5,587,396; Smith, which is hereby incorporated by reference, teaches a method of ameliorating cellulite by disrupting the barrier function of the stratum corneum that topically applied treatments for cellulite are shown by comparative data to effect structural improvements in cellulite-afflicted thigh area tissues including skin-thickening, thigh-firming and thigh-reduction. The disclosed treatments disrupt the skin's water barrier and elevate trans-disclosed treatments disrupt the skin's water barrier and elevate trans-epidermal water loss (TEWL) for extended periods of weeks or months and include methods of mechanical or solvent action, for example, tape stripping, or acetone washes. Preferred treatments use creams with active ingredients such as lactic acid to elevate TEWL, a retinoid, preferably vitamin A palmitate to disrupt barrier rebuilding and prolong elevation of TEWL levels, and a cerebroside to inhibit lipid synthesis and intensify the TEWL elevation. Diuretics, for immediate esthetic improvements, anti-irritants and anti-oxidants for irritation control are optional ingredients.

U.S. Pat. No. 5,051,449; Kligman, teaches a method for retarding and reversing cellulite comprises topically applying to human skin a retinoid in an amount and for a period of time effective to retard or reverse cellulite where said amount is insufficient to be excessively irritating. The method preferably uses retinoic acid in an emollient vehicle and is hereby incorporated by reference.

A product that could help stimulate the capillary blood flow would be beneficial to help reduce the appearance of cellulite.

### SUMMARY OF THE INVENTION

The present invention is a powerful vasodilatation and transdermal penetration system, which quickly penetrates the dermis of the skin and deposits beneficial ingredients at the subcutaneous layer, which then stimulates the capillary blood flow to fat cells and increases the metabolizing process of fat cells and cellulite, thereby reducing the size of fat cells, thus creating a smoother appearance to the surface of the skin as well as reducing the appearance of the size of the affected area.

### DETAILED DESCRIPTION OF THE EMBODIMENTS

Xanthines are a group of natural compounds that the body makes daily. Xanthine is a yellow-white crystalline compound found in blood and urine and in some plants. It is the precursor of uric acid, C5H4N4 O2. They are by products of tissue breakdown and have many functions in the body. One of the actions of the xanthines is to inhibit an enzyme called phosphodiesterase. The enzyme phosphodiesterase destroys cyclic adenosine monophosphate, which stimulate lipolytic action. By inhibiting the phosphodiesterase, the xanthines prolong the action of cyclic adenosine monophosphate, thereby increasing lipolytic action. For cellulite, this process is very important since the stored fat must be mobilized at specific sites of cellulite in order to be effective. Lipolytic action is a metabolic process in where raw fat that cannot be absorbed are broken down. Derivatives of xanthines include, but are not limited to caffeine and theophylline.

Caffeine, heavily consumed in coffee, tea and some cola drinks, has been shown in other studies to prompt mental alertness in many people.

A benefit of the topical use of caffeine anhydrous on the skin is a stimulatory effect under the surface of the skin.

The main benefit for the present invention is the reduction in water retention of the fat cells. Xanthines will decrease, even temporarily eliminate the sub-cutaneous water associated with cellulite, as well as increase the metabolism of fat, or fat burning. Topical caffeine also reduces facial and body puffiness. Caffeine is an astringent and antioxidant that strongly stimulates microcirculation. It has a lifting and tightening effect as well, which makes it a beneficial choice for anti-cellulite treatments. Other studies claim that caffeine stimulates lipolysis, or fat burning, and increases metabolism to burn fat faster. Caffeine is present in the current invention in amounts of 0.5% to 10.0%.

Theophylline is a xanthine derivative. When theophylline is combined with ethylene diamine in anhydrous alcohol, it forms the compound aminophylline, a smooth muscle relaxant. Theophylline does cause inhibition of the phosphodiesterase with resultant increase in intracellular adenosine monophosphate. It has been suggested that theophylline inhibits the activity of certain hormones that cause a woman's body to store fat and therefore release intracellular triglycerides. It is thought that xanthine derivatives block the receptors for these fat storage hormones on the fat cell causing the cell to release instead of retain its triglyceride contents. Other proposed mechanisms of action include translocation of intracellular calcium, prostaglandin antagonism, stimulation of catecholamines endogenously, and an inhibition of cyclic guanacine monophosphate metabolism, and possibly an adenosine receptor antagonism. Theophylline and xanthine derivatives are a necessary component for the present invention. Theophylline is present in the current invention in weight percentages of 0.01% to 15.0%.

Niacin is synonymous to nicotinic acid and a component of the vitamin B complex and is essential for metabolic processes in each cell of the body. Deficiency in Niacin can reduce cellular functions in the body. Niacin can function as a vasodilator. It is suggested that Niacin can help to widen dilation of blood vessels. Topical application of niacin is beneficial to the present invention to enhance blood circulation and stimulate the metabolism. Vasodilation is also a necessary component of the present invention as it is the mechanism of transdermal penetration, which deposits the beneficial ingredients of the topical formulation, allowing the invention to penetrate the stratum corneum and deposit the xanthine derivatives below the application site, thereby creating a stimulatory effect, thereby reducing puffiness and water retention on cellulite. Niacin is a necessary component of the present invention to vasodilate the capillaries which stimulate blood flow to pump lymph fluid. Niacin is present in the current invention in weight percentage ranges of 0.01% to 5.0%.

Methylsulfonylmethane is a naturally occurring, organic sulfur containing compound and is a precursor to dimethyl sulfoxide. Dimethyl sulfoxide has been used extensively in the animal medicine industry as a transportation vehicle for the delivery of drugs without the use of a syringe or needle. Methylsulfonylmethane is found in small amounts throughout nature and has been detected in small amounts in the blood and urine of humans. Animal studies have shown that sulfur from oral supplements of methylsulfonylmethane is incorporated into body proteins. Methylsufonylmethane is more tolerated to human use than dimethyl sulfoxide. Methylsulfonylmethane is present in the current invention in weight percentage ranges of 0.5% to 15.0%.

The present invention utilizes both the methylsulfonylmethane and the niacin as a novel and unique transdermal penetration system to vasodilate, thereby widening the pores of the skin allowing transport of the beneficial ingredients at the site of application to quickly reduce the appearance of cellulite.

Dimethicone, a silicone polymer is a silica that is sourced from sand. In its simplest form is polydimethylsiloxane, also known as silicone oil, but now more commonly called by its INCI name dimethicone. It was originally used for its waterproofing properties in barrier products, which protect the skin. Used at a rate of 1.0% to 30.0% dimethicone conforms to the FDA's Tentative Final Monograph on OTC Skin Protectants, the Federal Register/Vol. 54, No. 190/Tuesday, Oct. 3, 1989/Proposed Rules, Department of Health and Human Services, Food and Drug Administration, 21 CFR Part 347, Skin Protectant Drugs For Over-The-Counter Human Use states that on page 40815 under summary of active ingredient categories under skin protectant active ingredients table, dimethicone and glycerin are listed as a category 1 skin protectants. Glycerin and dimethicone are necessary components of the present invention. The present invention meets the FDA proposed rules as a skin protectant. Dimethicone and glycerin are present in the current invention in range percentages of 0.5% to 20.0%.

The typical formula with weight percentages are as follows:

| Wt % | Ingredient |
|---|---|
| 2.0-10.0 | Primrose Oil |
| 2.0-10.0 | Grapeseed Oil |
| 2.0-10.0 | Finsolv |
| 1.5-5.0 | Steareth-20 |
| 0.5-5.0 | Steareth-2 |
| 1.0-10.0 | Dimethicone |
| 0.75-5.0 | Polysorbate 60 |
| 0.5-15 | PEG-40 Stearate |
| 3.0-8.0 | Cetyl Alcohol |
| 2.0-10.0 | Glycerin |
| 1.0-5.0 | Myristal Myristate |
| 1.0-5.0 | Isopropyl Lanoate |
| 4.0-40.0 | Glycerin |
| 0.35-2.0 | Phenoxyethanol |
| 0.35-2.0 | Dimethyl Dimethyl Hydantoin |
| 0.70-2.0 | Xanthan Gum |
| 57.85-q.s. | Water |
| 3.5-10.0 | Caffeine |
| 4.0-10.0 | MSM |
| 0.50-5.0 | Niacin |
| 2.0-10.0 | Aloe Vera |
| 0.1-10.0 | Theophylline Blend; |
| | (90.0% water and 10.0% |
| | Theophylline) |

Various ingredients can be used in the formulation of the present invention, such as viscosity modifiers, which consist of waxes and gums. Viscosity modifiers adjust the base of the formulation, which dictate whether the consistency of the finished product is to become a topical formulation such as a cream, an ointment, a gel, a lotion, a spray, an enhanced oil and the like.

Waxes as viscosity modifiers can be selected from the group comprising, cetyl alcohol, PEG-40, stearic acid, beeswax, synthetic wax, behenyl alcohol, cetearyl alcohol, steareth derivatives, polyacrylates and combinations thereof. Waxes also act as barriers, for example, the thicker the product the higher content of waxes, the thicker the barrier layer. Viscosity modifiers are present in the current invention in weight percentage amounts of 0.5% to 30.0%.

Gums as viscosity modifiers can be selected from the group comprising xanthan gum, cellulose gums, guar gum and guar derivatives, gellan gum and combinations thereof. Gums not only act as a viscosity modifier, but also as a suspension agent for suspending and distributing the active ingredients efficiently within the solution. Gums are present in the current invention in weight percentage amounts of 0.01% to 5.0%.

Moisturizers and emollients in various degrees are present in the current invention and can be selected from the group comprising primrose oil, myristal myristate, hyaluronic acid, lanolin and lanolin derivatives, glycerin, squalene, jojoba oil, d-alpha tocopherol, C-12-C15 branched alkyl ester, propylene glycol, mineral oil, petrolatum; aloe vera and combinations thereof. Moisturizers help to alleviate dry skin and ease the appearance of fine lines and wrinkles. Moisturizers are present in the current invention in weight percentage amounts of 1.0% to 40.0%.

Natural and synthetic oils in various degrees are also incorporated into the present invention and can be selected from the group; evening primrose, primrose, squalene, grapeseed, apricot seed, mineral oil, almond, sesame, olive, peanut, soybean and combinations thereof. Oils natural and synthetic are present in the current invention in weight percentages of 2.0-35.0%.

Emulsifiers are present in the current invention and can be selected from the group comprising, glyceryl monostearate, polysorbate 20, polysorbate 60, polysorbate 80 and combinations thereof. Emulsifiers are present in the current invention in weight percentage amounts of 0.5 to 10.0%.

Preservatives are present in the current invention and can be selected from the group comprising methylparaben, propylparaben, paraparaben, imidyazadonyl urea, dimethyl dimethyl hydantoin and combinations thereof. Preservatives are present in the current invention in weight percentages of 0.01 to 5.0%.

Fragrances are optional can be added to the formula of the present invention and can be water or oil base, natural, synthetic or combinations thereof. Fragrances can be added to the present invention in weight percentages of 0.01% to 3.0%.

The present embodiments are therefore to be considered in all respects as illustrative and not restrictive, the scope of the formulation being indicated by the appended claims rather than by the foregoing description. All changes, which come within the meaning and range of equivalency of the claims, are intended to be embraced herein.

### Clinical Studies

### Protocol

The inventors sponsored a clinical study at a reputable dermatological laboratory. The results are as follows.

Twenty-five female subjects with ages ranging from 25 to 45 with visible signs of cellulite on their upper thigh participated and completed a sixty days product efficacy in-use-study. Subjects applied the treatment product to their thigh area 2 to 4 times a day. The subjects visited the test center at Day 0, Day 30 and Day 60 for the following five evaluations made by trained technicians: 1) Body Weight; 2) Skin Texture; 3) Thigh Circumference; 4) Skin Layers Thickness; and 5) Skin Firmness.

Close-up photographs of five selective subjects at their thighs area will be taken at baseline and Day 60.

To ensure that all subjects were at a baseline value and to factor out differences in effects of current skin care regimens, a one-week dry out period preceded the study. On week 2 subjects visited the test center were given a blind bar of soap to use exclusively for cleansing purposes as often as they chose. They were also instructed not to use any moisturizer, sunscreen, or liquid make-up on their thigh during this phase of the study. They also were instructed to avoid excessive Lw exposure and tanning salons.

On Day 0 all subjects visited the test center, in the AM without product applied to their thigh and given instructions on how to use the test product as well as a daily diary to record their use of the product and observations. Subjects were instructed to cleanse with the soap provided during the dry out period. Subjects then applied the product liberally to their entire thigh area 2 to 4 times a day throughout the treatment period. All subjects to be evaluated on Day 0 and again at Day 30 and Day 60 for the evaluation of: 1) measurement of the body weight; 2) clinical evaluation of the appearance of the skin; 3) measurement with tape measure of the circumferences of thighs; 4) skin thickness and thickness of the adiposities with ultrasound; and 5) skin firmness with ballistometry. Close-up photographs of the thigh area of five selective subjects were taken at baseline and at Day 60.

The body weight of each volunteer will be measured with a weighing scale at the beginning of Day 1, again at Day 30 and again at Day 60. The averages calculated and changes in the averaged body weight with time of treatment was determined.

A clinical assessment of the global, visual and tactile aspects of localized adiposity and cellulite will be carried out by the investigator using an assessment scale as a reference for scoring the skin condition. The scale is a five-point scale with each numbering point clearly differentiating the different skin conditions, as seen in Table 1 below. After careful examination of the subjects skin condition the investigator will select a score, which closely resemble both the tactile and visual quality of the subjects skin. The score from each subject at different time points will be collected, tabulated and averaged.

Table 1. Five Point Scale Scoring System.

Grade Visual & Tactile Grading of Localized Adiposities and of Cellulite

0 No excess localized adipose accumulation, smooth skin.
1 Slight localized adipose accumulation, slight orange peel appearance to the skin, very small barely noticeable cellulite bumps, slight mattress-like skin appearance.
2 Moderate localized adipose accumulation, perceptible orange pee appearance to the skin, clearly felt bumps, perceptible mattress-like skin appearance.
3 Evident localized adipose accumulation, moderate orange peel appearance to the skin, moderate bumps, moderate mattress-like skin appearance.
4 Severe localized adipose accumulation, marked orange peel appearance to the skin, Heavy, deep bumps, severe mattress-like skin appearance

The measurement of the thigh circumference is carried out with a tape measure draped snugly on skin without exerting pressure. Sites of measurements are marked with magic marker are identified by obtaining the distance of the sites to the floor. Measurements were also made before treatment started as well as at different evaluation time points.

The thickness of the fatty tissue layer of the skin was measured by Ecographic evaluation of the skin via an Ultrasound scanner. For this, a 20 Mhz ultrasound scanner (Dermascan C, Cortex Technology, Denmark) following standardized data gathering methods was used. This instrument enabled the production of images representing a cross-section of the skin and by computer image analysis, so the thickness of the fatty tissue layer can be measured.

The Ballistometer measures skin firmness by dropping a pendulum to the skin surface and the resultant bounding pattern of the pendulum is analyzed by a computer to determine skin firmness. Measurement with the Ballistometer is achieved by measuring the rebounce pattern of the device using a standardized rubber foam pad and adjusting the instrument to reproduce the same bouncing pattern. Each test day the unit is calibrated and a reading made on the defined area of each subject. Although numerous forms of analysis of Ballistometry data can be made, the ratio of the height of the first (H1) and second rebound peaks (H2) is used as a measure of skin firmness. The data was obtained following standardized gathering methods.

### Clinical Study Results

The body weight of each panelist was determined with a weighing scale, at the beginning Day 0 of the study and at Day 30 and Day 60 post-treatment. The averages were calculated and the relative changes in the averaged weight of subjects before treatment and at various evaluation time points were determined. The results are shown in Table 1. As the results indicate; there is a small but statistically significant decrease (−4.2%) in body weight after 30 days of product treatment. With the continuation of treatment to 60 days, a more significant and larger decrease in body weight was observed (−5.8%).

TABLE 1
Changes In Body Weight.

| Subject # | Day 0 | Day 30 | Day 60 |
|---|---|---|---|
| 1 | 83 | 81 | 78 |
| 2 | 64 | 63 | 62 |
| 3 | 61 | 58 | 57 |
| 4 | 67 | 66 | 68 |
| 5 | 71 | 65 | 62 |
| 6 | 94 | 91 | 89 |
| 7 | 67 | 65 | 65 |
| 8 | 78 | 71 | 68 |
| 9 | 68 | 63 | 61 |
| 10 | 66 | 66 | 69 |
| 11 | 62 | 60 | 59 |
| 12 | 68 | 67 | 69 |
| 13 | 64 | 60 | 58 |
| 14 | 72 | 71 | 68 |
| 15 | 85 | 81 | 78 |
| 16 | 61 | 62 | 60 |
| 17 | 81 | 77 | 75 |
| 18 | 81 | 78 | 79 |
| 19 | 73 | 69 | 67 |
| 20 | 62 | 58 | 57 |
| 21 | 69 | 64 | 63 |
| 22 | 64 | 60 | 59 |
| 23 | 78 | 74 | 70 |
| 24 | 82 | 80 | 83 |
| 25 | 65 | 61 | 59 |
| Average | 71.44 | 68.44 | 67.32 |
| S.D. | 8.926 | 8.689 | 8.745 |
| t | | 7.924 | 6.204 |
| p* | | <0.0001 | <0.0001 |
| % | | −4.2% | −5.8% |
| Change | | | |

*Paired T Test at 95% Confidence limited

A clinical assessment of the global, visual and tactile aspects of localized adiposity and cellulite was carried out by the investigator using an assessment scale as reference for scoring. The scale is a numerical five-point scale with each numbering point clearly differentiating the different condition of the skin. After careful examination of the subject skin condition, the investigator selected a score, which closely resembled the skin condition. The scores from different subjects at each visit were collected, tabulated and presented in Table 2. As the results indicate, the treatment showed significant improvement in the tactile and visual aspects of the localized adiposities and in the cutaneous imperfections caused by cellulite throughout the course of the study. Such improvement included the decrease of the adipose accumulations, in the orange peel and mattress-like skin appearance and in the presence of nodosity. Improvements were seen, as early as Day 30 of treatment and optimal results were observed at 60 days post-treatment.

TABLE 2
Clinical Evaluation of the Appearance of the Skin.

| Subject # | Day 0 | Day 30 | Day 60 |
|---|---|---|---|
| 1 | 4 | 3 | 3 |
| 2 | 2 | 1 | 1 |
| 3 | 2 | 1 | 1 |
| 4 | 3 | 2 | 1 |
| 5 | 3 | 2 | 2 |
| 6 | 4 | 3 | 3 |
| 7 | 3 | 2 | 2 |
| 8 | 4 | 3 | 2 |
| 9 | 3 | 3 | 2 |
| 10 | 2 | 2 | 1 |
| 11 | 2 | 1 | 1 |
| 12 | 3 | 2 | 2 |
| 13 | 2 | 2 | 2 |
| 14 | 3 | 3 | 3 |
| 15 | 4 | 3 | 3 |
| 16 | 2 | 2 | 2 |
| 17 | 3 | 3 | 3 |
| 18 | 4 | 4 | 3 |
| 19 | 3 | 3 | 2 |
| 20 | 2 | 2 | 2 |
| 21 | 3 | 2 | 2 |
| 22 | 2 | 2 | 1 |
| 23 | 4 | 3 | 2 |
| 24 | 4 | 3 | 3 |
| 25 | 3 | 2 | 2 |
| Average | 2.96 | 2.36 | 2.04 |
| S.D. | 0.790 | 0.757 | 0.735 |
| t | | 6.000 | 8.048 |
| p* | | <0.0001 | <0.0001 |
| % | | 20.3% | 31.1% |
| Change | | | |

*Paired T Test at 95% Confidence Limited

The measurements of thigh circumference were carried out with a tape measure draped snugly on skin without exerting pressure. Sites of measurements were marked with magic marker and were identified by obtaining the distance of the sites to the floor. Results are presented in Table 3. As the results indicate, the test treatment showed statistically significant reductions in thigh circumference after 30 days of treatment and the thigh circumference continued to reduce for up to 60 days of treatment. Reductions of 3.0% and 6.2% were found at day 30 and day 60 post-treatment respectively.

TABLE 3
Circumference Measurements of the
Thighs with a Tape Measure.

| Subject # | Day 0 | Day 30 | Day 60 |
|---|---|---|---|
| 1 | 63.4 | 61.7 | 59.4 |
| 2 | 51.7 | 50.8 | 48.7 |
| 3 | 50.8 | 48.9 | 46.8 |
| 4 | 52.7 | 50.2 | 49.3 |
| 5 | 54.9 | 52.3 | 50.2 |
| 6 | 65.6 | 63.4 | 61.8 |
| 7 | 53.7 | 50.2 | 48.6 |
| 8 | 58.4 | 56.7 | 54.3 |
| 9 | 55.7 | 54.3 | 51.2 |
| 10 | 54.2 | 54.8 | 52.7 |
| 11 | 53.3 | 52.6 | 51.9 |
| 12 | 55.7 | 53.7 | 52.4 |
| 13 | 50.8 | 48.2 | 47.3 |
| 14 | 57.2 | 55.6 | 52.1 |
| 15 | 64.5 | 61.1 | 60.8 |
| 16 | 50.6 | 48.7 | 48.6 |
| 17 | 62.7 | 61.3 | 58.5 |
| 18 | 60.4 | 58.4 | 55.9 |
| 19 | 59.8 | 58.8 | 56.4 |
| 20 | 48.7 | 48.1 | 47.3 |
| 21 | 51.6 | 50.7 | 48.5 |
| 22 | 49.8 | 48.2 | 47.7 |
| 23 | 55.2 | 54.7 | 52.6 |
| 24 | 59.6 | 57.6 | 54.8 |
| 25 | 50.3 | 48.4 | 47.1 |
| Average | 55.65 | 53.98 | 52.20 |
| S.D. | 4.954 | 4.832 | 4.516 |
| t | | 9.197 | 15.435 |
| p* | | <0.0001 | <0.0001 |
| % | | −3.0% | −6.2% |
| Change | | | |

*Paired T Test at 95% Confidence Limited

An ultrasound site measurement of upper thighs teaches that subjects saw an overall reduction of the skins fatty layer thickness resulting in changes of 6.4% on day 30 to 8.2% after 60 days as seen in table 4.

TABLE 4
Skin Fatty Layer Thickness.

| Subject # | Day 0 | Day 30 | Day 60 |
|---|---|---|---|
| 1 | 15.216 | 14.021 | 13.813 |
| 2 | 13.147 | 12.462 | 12.165 |
| 3 | 13.185 | 12.648 | 12.215 |
| 4 | 13.622 | 12.021 | 11.748 |
| 5 | 14.258 | 13.964 | 13.659 |
| 6 | 15.326 | 14.846 | 14.325 |
| 7 | 12.876 | 11.523 | 11.623 |
| 8 | 14.105 | 13.174 | 12.946 |
| 9 | 13.629 | 12.036 | 11.847 |
| 10 | 13.215 | 13.079 | 12.562 |
| 11 | 12.411 | 11.964 | 11.417 |
| 12 | 13.062 | 12.543 | 12.091 |
| 13 | 12.956 | 12.629 | 12.706 |
| 14 | 14.025 | 13.748 | 13.534 |
| 15 | 14.964 | 13.626 | 13.629 |
| 16 | 13.177 | 12.528 | 12.573 |
| 17 | 14.513 | 13.021 | 12.626 |
| 18 | 13.976 | 12.462 | 12.154 |
| 19 | 13.205 | 12.915 | 12.638 |
| 20 | 12.513 | 11.627 | 11.849 |
| 21 | 12.764 | 11.943 | 11.636 |
| 22 | 12.059 | 11.021 | 10.956 |
| 23 | 13.754 | 12.585 | 12.543 |
| 24 | 14.962 | 13.496 | 12.754 |
| 25 | 12.053 | 11.252 | 11.153 |
| Average | 13.56 | 12.69 | 12.45 |
| S.D. | 0.942 | 0.929 | 0.861 |
| t | | 9.206 | 11.135 |
| p* | | <0.0001 | <0.0001 |
| % | | −6.4% | −8.2% |
| Change | | | |

*Paired T Test at 95% Confidence Limited

The ballistometry site assessment of the thighs teaches that subjects saw an overall change of skin firmness resulting in changes of 16.3% on day 30 to 24.4% after 60 days as seen in table 5, indicating skin becomes firmer with use.

TABLE 5
Skin Ballistometry Objective Assessment.

| Subject # | Day 0 | Day 30 | Day 60 |
|---|---|---|---|
| 1 | 5.66 | 5.16 | 4.85 |
| 2 | 5.13 | 4.52 | 3.97 |
| 3 | 4.87 | 4.36 | 4.08 |
| 4 | 5.26 | 4.51 | 3.94 |
| 5 | 5.48 | 4.23 | 3.62 |
| 6 | 5.78 | 4.16 | 3.73 |
| 7 | 5.12 | 4.24 | 3.95 |
| 8 | 5.52 | 4.31 | 3.81 |
| 9 | 5.17 | 4.78 | 4.25 |
| 10 | 5.08 | 4.46 | 4.03 |
| 11 | 4.95 | 4.18 | 3.76 |
| 12 | 5.26 | 4.03 | 3.56 |
| 13 | 4.87 | 4.05 | 3.84 |
| 14 | 5.39 | 4.24 | 3.76 |
| 15 | 5.57 | 4.61 | 4.25 |
| 16 | 5.08 | 4.46 | 4.04 |
| 17 | 5.61 | 5.39 | 4.81 |
| 18 | 5.52 | 4.37 | 4.14 |
| 19 | 5.34 | 4.16 | 3.62 |
| 20 | 5.15 | 4.25 | 3.86 |
| 21 | 5.27 | 4.73 | 4.52 |
| 22 | 4.86 | 4.38 | 3.74 |
| 23 | 5.48 | 4.03 | 3.68 |
| 24 | 5.49 | 4.32 | 3.97 |
| 25 | 4.82 | 4.27 | 3.86 |
| Average | 5.27 | 4.41 | 3.99 |
| S.D. | 0.276 | 0.328 | 0.338 |
| t | | 11.903 | 17.089 |
| p* | | <0.0001 | <0.0001 |
| % | | 16.3% | 24.4% |
| Change | | | |

*Paired T Test at 95% Confidence Limited

### STUDY CONCLUSION

The study evaluated the clinical efficacy and the safety of use of an anti-cellulite treatment on 25 female subjects. The laboratory and clinical testings in the study provided results proving that the product tested was effective in providing anti-cellulite effects. Consequently, Day 30 of product treatment was shown to provide statistically significant reduction of body weight, thigh circumference as well as the thickness of adipose tissue layer. Additionally, improvement in the cellulite skin condition was observed whereby the orange peel and mattress like appearance of the skin was significantly reduced. Skin firmness was also found to increase significantly. With treatment continued, all of the aforementioned clinical parameters continued to improve with much greater anti-cellulite effect seen after Day 60 of treatment.

Overall, the present invention provided significant improvements in many skin properties as measured objectively and instrumentally by the investigator. Moreover, in spite of the profound efficacy observed with the treatment of the product, no complaints of skin irritation or sensitization were reported.

While only a few embodiments of the formulation composition have been disclosed in the above detailed description, the formulation is not limited thereto but is susceptible to various changes without departing from the scope of the formulation.

The formulation composition may be embodied in many forms without departing from the spirit or essential characteristics of the formulation.

1. A composition for the treatment of cellulite or fatty deposits under the skin comprising: Caffeine 3.5-10.0 wt %; Theophylline 0.500-10.0 wt %; Niacin 0.01-5.0 wt %; Methylsulfonylmethane 4.0-10.0 wt %; Vasodilators for Transdermal Penetration 4.5%-10.0 wt %; Xanthine Derivatives 7.5-20.0 wt %; Phenoxyethanol and Dimethyl Dimethyl Hydantoin 0.70-4.0 wt %; Emollients and Moisturizers 10.0-50.0 wt %; Gums 0.01-5.0 wt %; Emulsifiers 5.25-40.0 wt %; and Skin Protectants 0.01-50.0 wt %.

2. The composition of claim 1, wherein the vasodilators for the transdermal penetration system is selected from the group methylsulfonylmethane, niacin, and combinations thereof.

3. The composition of claim 1, wherein the xanthine stimulants are selected from the group caffeine, theopylline and combinations thereof.

4. A composition of claim 1, wherein the skin protectant system is selected from the group glycerin, dimethicone and combinations thereof.

5. A topical formulation to reduce body weight by the treatment of cellulite or fatty deposits under the skin comprising: Caffeine 3.5-10.0 wt %; Theophylline 0.500-10.0 wt %; Niacin 0.01-5.0 wt %; Methylsulfonylmethane 4.0-10.0 wt %; Vasodilators for Transdermal Penetration 4.5%-10.0 wt %; Xanthine Derivatives 7.5-20.0 wt %; Phenoxyethanol and Dimethyl Dimethyl Hydantoin 0.70-4.0 wt %; Emollients and Moisturizers 10.0-50.0 wt %; Gums 0.01-5.0 wt %; Emulsifiers 5.25-40.0 wt %; and Skin Protectants 0.01-50.0 wt %.

6. The topical formulation of claim 2, wherein the vasodilators for the transdermal penetration system is selected from the group methylsulfonylmethane, niacin, and combinations thereof.

7. The topical formulation of claim 2, wherein the xanthine stimulants are selected from the group caffeine, theopylline and combinations thereof.

8. A composition of claim 2, wherein the skin protectant system is selected from the group glycerin, dimethicone and combinations thereof.

9. A topical formulation to improve the overall texture of the skin by the treatment of cellulite or fatty deposits comprising: Caffeine 3.5-10.0 wt %; Theophylline 0.500-10.0 wt %; Niacin 0.01-5.0 wt %; Methylsulfonylmethane 4.0-10.0 wt %; Vasodilators for Transdermal Penetration 4.5%-10.0 wt %; Xanthine Derivatives 7.5-20.0 wt %; Phenoxyethanol and Dimethyl Dimethyl Hydantoin 0.70-4.0 wt %; Emollients and Moisturizers 10.0-50.0 wt %; Gums 0.01-5.0 wt %; Emulsifiers 5.25-40.0 wt %; and Skin Protectants 0.01-50.0 wt %.

10. The topical formulation of claim 3, wherein the vasodilators for the transdermal penetration system is selected from the group methylsulfonylmethane, niacin, and combinations thereof.

11. The topical formulation of claim 3, wherein the xanthine stimulants are selected from the group caffeine, theopylline and combinations thereof.

12. A composition of claim 3, wherein the skin protectant system is selected from the group glycerin, dimethicone and combinations thereof.

13. A topical formulation to reduce thigh circumference by the treatment of cellulite or fatty deposits comprising: Caffeine 3.5-10.0 wt %; Theophylline 0.500-10.0 wt %; Niacin 0.01-5.0 wt %; Methylsulfonylmethane 4.0-10.0 wt %; Vasodilators for Transdermal Penetration 4.5%-10.0 wt %; Xanthine Derivatives 7.5-20.0 wt %; Phenoxyethanol and Dimethyl Dimethyl Hydantoin 0.70-4.0 wt %; Emollients and Moisturizers 10.0-50.0 wt %; Gums 0.01-5.0 wt %; Emulsifiers 5.25-40.0 wt %; and Skin Protectants 0.01-50.0 wt %.

14. The topical formulation of claim 4, wherein the vasodilators for the transdermal penetration system is selected from the group methylsulfonylmethane, niacin, and combinations thereof.

15. The topical formulation of claim 4, wherein the xanthine stimulants are selected from the group caffeine, theopylline and combinations thereof.

16. A composition of claim 4, wherein the skin protectant system is selected from the group glycerin, dimethicone and combinations thereof.

17. A topical formulation to reduce the skin fatty layer thickness by the treatment of cellulite or fatty deposits comprising: Caffeine 3.5-10.0 wt %; Theophylline 0.500-10.0 wt %; Niacin 0.01-5.0 wt %; Methylsulfonylmethane 4.0-10.0 wt %; Vasodilators for Transdermal Penetration 4.5%-10.0 wt %; Xanthine Derivatives 7.5-20.0 wt %; Phenoxyethanol and Dimethyl Dimethyl Hydantoin 0.70-4.0 wt %; Emollients and Moisturizers 10.0-50.0 wt %; Gums 0.01-5.0 wt %; Emulsifiers 5.25-40.0 wt %; and Skin Protectants 0.01-50.0 wt %.

18. The topical formulation of claim 5, wherein the vasodilators for the transdermal penetration system is selected from the group methylsulfonylmethane, niacin, and combinations thereof.

19. The topical formulation of claim 5, wherein the xanthine stimulants are selected from the group caffeine, theopylline and combinations thereof.

20. A composition of claim 5, wherein the skin protectant system is selected from the group glycerin, dimethicone and combinations thereof.

21. A topical formulation to increase skin firmness by the treatment of cellulite or fatty deposits comprising: Caffeine 3.5-10.0 wt %; Theophylline 0.500-10.0 wt %; Niacin 0.01-5.0 wt %; Methylsulfonylmethane 4.0-10.0 wt %; Vasodilators for Transdermal Penetration 4.5%-10.0 wt %; Xanthine Derivatives 7.5-20.0 wt %; Phenoxyethanol and Dimethyl Dimethyl Hydantoin 0.70-4.0 wt %; Emollients and Moisturizers 10.0-50.0 wt %; Gums 0.01-5.0 wt %; Emulsifiers 5.25-40.0 wt %; and Skin Protectants 0.01-50.0 wt %.

22. The topical formulation of claim 6, wherein the vasodilators for the transdermal penetration system is selected from the group methylsulfonylmethane, niacin, and combinations thereof.

23. The topical formulation of claim 6, wherein the xanthine stimulants are selected from the group caffeine, theopylline and combinations thereof.

24. A composition of claim 6, wherein the skin protectant system is selected from the group glycerin, dimethicone and combinations thereof.
